(19) United States
(12) Patent Application Publication
(10) Pub. No.: US 2006/0247621 A1
(43) Pub. Date: Nov. 2, 2006

(54) Hybrid interlocking proximal femoral fracture fixation device and an operative technique of introducing the same

(76) Inventor: Marc Waisman, Kyriat Bialik (IL)

Correspondence Address: Angenehm Law Firm. Ltd., P.O. Box 48755, Coon Rapids, MN, 55448-0755, US

(21) Appl. No.: 11/476,466

(22) Filed: Jun. 28, 2006

Related U.S. Application Data
(63) Continuation-in-part of application No. 10/794,693, filed on Mar. 5, 2004, now Pat. No. 7,094,236.
(60) Provisional application No. 60/457,216, filed on Mar. 25, 2003.

(51) Int. Cl. A61B 17/56

(52) U.S. Cl. 606/60

(57) ABSTRACT

The present invention provides unique and novel internal fixation for fixing intracapsular and extracapsular fracture of a femoral neck including subtrocanteric fractures while using minimal invasive procedure. The fixation comprises a hollow spaced or full tubular member having a sharp end and a blunt end that is adapted to be implanted while slightly crossing the fracture line. The tubular member is provided with three passages that extend from the sharp end to the blunt end. Three lag screws are adapted to pass through the passages and extend outwardly beyond said sharp end while thread that is provided in the lag screws so as to allow compression of fragments of the bone. The lag screws are provided with screw heads so as to prevent the screws from being fully inserted into the passages and through the trochanteric cortical bone. The internal fixation can be incorporated into an external-interlocking fixation apparatus that comprises the tubular member laterally provided with bores on opposite sides of the tubular member wherein pin screws are adapted to be inserted through the bores so as to interlock the tubular member and additional pin screw that are nailed in a distal femoral fragment. A connecting member is adapted to secure the pin screws together so as to assure stability of the tubular member within the bone and to allow reduction, correction and fixation in the intra and post-operative period.

### FIELD OF THE INVENTION

This present application claims the benefit of earlier U.S. provisional patent application Ser. No. 60/457,216 filed on Mar. 25, 2003 by Waisman Marc and entitled “Hybrid Interlocking Proximal Femoral Fracture Fixation” and is a continuation-in-part of U.S. patent application Ser. No. 10/794,693 filed on 5^{th} of March 2004, claiming priority therefrom.

The present invention relates to fracture fixation. More particularly, the present invention relates to hybrid interlocking proximal femoral fracture fixation and a method of introducing such a device in a minimal invasive manner.

### BACKGROUND OF THE INVENTION

Internal fixation with nails and plates is a well-known surgical procedure used in orthopaedics and traumatology for stabilization of proximal femoral fractures. This procedure is considered as a classical open major surgery carrying out several possibilities of serious complications. It was considered in the past that rigidity of the fracture fixation site is advantageous, therefore, many of the available internal fixation devices are built so as to eliminate all movements (except of sliding possible motion) at the fracture site. It is now generally accepted that some micro-movements at the fracture site are essential for better fracture healing and even for stimulating callus formation. However, this conception is not valid for intracapsular femoral neck fractures.

Internal fixation bears many disadvantages including the fact that the surgery is highly expensive and complex, which may be complicated by significant blood loss and infection. There is a lack of ability to perform post-operative re-fixation, the morbidity and mortality rates are high and as a consequence of the surgery, there is a prolonged hospitalization related to peri-operative complications. The death rates following internal fixation in cases of subcapital (intracapsular) fractures are intimidating: 3% in the hospital, 25% at one year and additional 40% at two years following the surgery. 30% experience avascular necrosis, 43% non-union and 50% experience peri-operative—postoperative local and systemic complications. The data is collected from Clinical Orthopaedics and Related Research 348:22-28, 1998; Clinical Orthopaedics and Related Research 399:119-128, 2002. These papers are incorporated herein as references; however, similar results were established and published in many other scientific reviewed publications. The consequences of intertrochanteric-pertrochanteric (extracapsular) fractures are no less frightening. 15% experience fixation failure, 10% dies at one year; 20% at two year, 20% complicated with infection, and 30% with mal-union. Similar consequences are found in subtrochanteric fractures.

External fixation using nails and screws connected to the femoral head, neck, and shaft through an external device provides the possibility to stabilize the fracture. This procedure is done using minimal invasive interventional surgery.

Clinical evidences clearly indicate that stabilization of a peritrochanteric femoral neck fracture by external fixation markedly reduces mortality, reduces the incidence of severe complications and improve fracture outcomes at the immediate postoperative time in comparison with the classical internal fixation. External fixation has other advantages such as decreased length of hospitalization and medical costs, reduces post-operative fracture pain, facilitates the access to the patient nursering care, reduces need for forced recumbency as well as risk of pressure sores, pulmonary embolism, pulmonary infection etc. External fixation is a safe and reliable method of achieving osseous stability in trochanteric femoral fractures. Generally, external fixation imparts versatility, ease to apply with minimal operative time, bleeding and tissue injury.

A percutaneous connection of a fractured upper part of the femur is disclosed in U.S. Pat. No. 5,429,641. Another example of an external trochanter splint is disclosed in U.S. Pat. No. 5,728,096. European patent application EP 0940124A1 teaches an external fixation device with changeable angle for trochanteric fractures. The devices that are disclosed herein as references as well as other similar devices for external fixation of trochanteric femoral fractures have many disadvantages and complications. One of the dangerous occurrences is the penetration of the neck screws into the acetabulum due to severe osteoporosis. Other disadvantages are hardware failure, the device is fixed in a lateral posterior bulging position that is uncomfortable, and there is an immense difficulty in the supine or sitting position of the patient.

It is a long felt need to provide an external device that eliminates the severe disadvantages of the available devices for external fixation of peritrochanteric fractures, which is one of the fixations that results in several complications.

As for the internal fixation, specially designed screws were developed, for example a screw member that is disclosed in PCT application published as WO00/67653, an intramedullary cavity nail disclosed in EP 0853923, or an anchor that is disclosed in US patent application 2002/0143333. Another commercially available fixation is sold by Fixano s.a. by the commercial name osteosynthesis of unstable femoral neck fractures by D.S.S. system (double sliding screws).

### SUMMARY OF THE INVENTION

It is an object of the present invention to provide a hybrid interlocking proximal femoral fracture fixation that enables minimal invasive fracture fixation. In this way, early callus formation in extracapsular fractures occurs.

It is another object of the present invention to provide an internal fixation for femoral neck intracapsular fractures that exhibits continuous compression by sliding properties.

It is yet another object of the present invention to provide a hybrid interlocking proximal femoral fracture fixation that combines a new and unique internal nail and an external non-rigid fixator, for extracapsular fractures.

An additional object of the present invention is to provide a fixation technique that is easy to use, and requires relatively short and minimal procedure for the surgeon.

It is therefore provided in accordance with one aspect of the present invention, an internal fixation device for fixing an intracapsular fracture of a femoral neck, comprising:
a nail-cage member having a sharp end and a blunt end;
at least one hollow space extending from said sharp end to said blunt end;
at least two passage extending from said sharp end to said blunt end;
at least two screws adapted to pass through said at least two passages and extend beyond said sharp end;
thread provided in a portion of said at least two screws that is adapted to extend beyond said sharp end;
screw head provided in each of said at least two screws so as to prevent said at least two screws from being fully inserted into said at least two passages;
whereby said nail-cage member is implanted in the bone crossing the fracture line by at least 1 to 2 milimeters, and said at least two screws are inserted through said at least two passages so that compression of fragments of the bone is maintained in order to facilitate the healing process.

Furthermore and in accordance with yet another preferred embodiment of the present invention, said nail-cage member is tube-like.

Furthermore and in accordance with yet another preferred embodiment of the present invention, said nail-cage member has a profile such as a circular, oval, triangular, or rectangular or others.

Furthermore and in accordance with yet another preferred embodiment of the present invention, nail-cage member is provided with holes.

Furthermore and in accordance with yet another preferred embodiment of the present invention, said at least one hollow space is filled with bone grafting materials so as to promote bone healing.

Furthermore and in accordance with yet another preferred embodiment of the present invention, said at least two passages are partially open.

Furthermore and in accordance with yet another preferred embodiment of the present invention, three lag screws are provided to correspond three passages that are provided in said nail-cage member.

Furthermore and in accordance with yet another preferred embodiment of the present invention, said nail-cage member is inserted to the femoral neck in an angle of preferably about 95-115 degrees in respect with an axial line of the femoral shaft so that an inferior screw of said three lag screws is positioned in a direction of an the inferior quadrant of the femoral head so as to slightly touch a the strong cortical bone of a calcar femori of the femoral neck.

Furthermore and in accordance with yet another preferred embodiment of the present invention, said three screws are adapted to penetrate an inferior quadrant of the femoral head, preferable distally of the teres ligament vascularity.

Furthermore and in accordance with yet another preferred embodiment of the present invention, said at least one hollow space is provided with bone or bone substitutes allowing bone grafting into said nail-cage member.

Furthermore and in accordance with yet another preferred embodiment of the present invention, said fixation device is further interlocked with external fixator.

It is also provided in accordance with yet another embodiment of the present invention, a method for fixing an intracapsular fracture of a femoral neck, the method comprising:
providing an internal fixator comprising
   a nail-cage member having a sharp end and a blunt end;
   at least one hollow space provided in said nail-cage member wherein said at least one hollow space extends from said sharp end to said blunt end;
   and at least two passage extending from said sharp end to said blunt end;
performing 2-4 cm long skin incision at the trochanter region after fracture reduction;
inserting said internal fixator to the femoral neck preferably in an angle of about 95-115 degrees in respect with an axial line of the femoral shaft so that an inferior passage of said at least two passages is adapted to be positioned in a direction toward the inferior quadrant of the femoral head so as to slightly touch a strong cortical bone of the calcar femori, wherein said nail-cage member is crossing the fracture line by at least 1 to 2 milimeters;
   providing at least two screws, each screw comprising:
      thread provided in a portion that is adapted to extend beyond said sharp end;
      screw head adapted to prevent the screw from being fully inserted into said nail-cage;
screwing said at least two screws so as to pass through said at least two passages and extend beyond said sharp end.

Furthermore and in accordance with yet another preferred embodiment of the present invention, the method further comprising inserting bone grafting materials to within said at least one hollow space.

Furthermore and in accordance with yet another preferred embodiment of the present invention, the method further comprising:
providing at least two bores in said nail-cage member and at least two opposite bores on opposite side of said nail-cage member;
interlocking at least two pin screws through the bores;
nailing at least two pin screws in a distal bone fragment;
providing a connecting member adapted to secure all pin screws together.

Furthermore and in accordance with yet another preferred embodiment of the present invention, the method further comprising correcting, reducing and fixating in intra and post-operative period.

### BRIEF DESCRIPTION OF THE FIGURES

In order to better understand the present invention and appreciate its practical applications, the following Figures are attached and referenced herein. Like components are denoted by like reference numerals.

It should be noted that the figures are given as examples and preferred embodiments only and in no way limit the scope of the present invention as defined in the appending Description and Claims.

FIG. 1 illustrate views of a nail-cage implant in accordance with a preferred embodiment of the present invention.

FIG. 2 illustrates an isometric view of a nail cage implant in accordance with another preferred embodiment of the present invention.

FIG. 3 illustrates an isometric view of the nail-cage implant fixed in an internal fixation in accordance with a preferred embodiment of the present invention.

FIG. 4 illustrates an isometric view of a hybrid interlocking proximal femoral fracture fixation in accordance with a preferred embodiment of the present invention, and its positioning in a femoral bone.

### DETAILED DESCRIPTION OF THE INVENTION AND THE FIGURES

The present invention provides a new and unique femoral fracture fixation that comprises an internal fixator and an external fixator. Basically, the internal fixator for an intracapsular fracture of a femoral neck comprises a tubular member having a sharp end and a blunt end. The tubular member can be a hollow space member or a solid member. At least one passage extending from the sharp end to the blunt is provided in the tubular member wherein the passages are adapted to accommodate lag screws. The screws are adapted to extent outwardly beyond said sharp end and into the bone so that compression of the fracture is maintained in order to facilitate the healing process, therefore, the portion of the screws that extend beyond the sharp end is provided with a thread. It is preferable to provide 3 passages and corresponding lag screws. The tubular member will be referred in this text also as a nail-cage. The nail-cage and the lag screws can be used as “stand alone” as a novel and unique internal fixator of intracapsular femoral neck fractures and as an alternative for the classic cannulated screws, allowing bone grafting into the nail-cage. The nail-cage and the lag screws are mainly intended for Garden 1-2 and 3 subcapital femoral fractures.

The nail-cage is inserted into the bone in a minimal invasive surgery. The nail-cage is inserted preferably through approximately 2-4 cm long skin incision after drilling the bone and forming a hole in the femoral neck that is adapted to receive the nail-cage. The external parts of the fixator are suitable to be removed in the out-patient follow up, without anesthesia. The external parts of the device are preferably made of titanium or other radiolucent materials such as aluminum 70 that is approved by FDA and are optionally disposable.

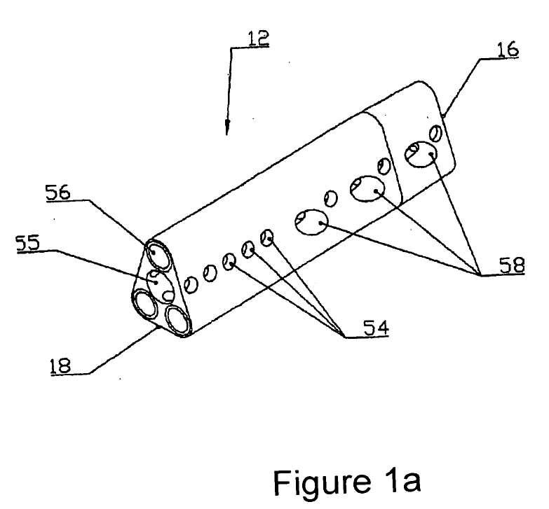

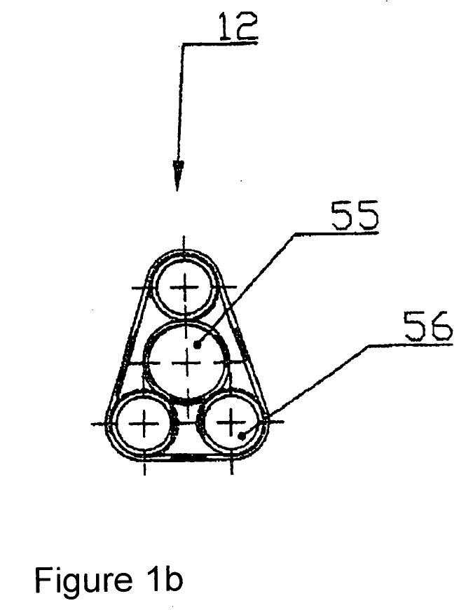

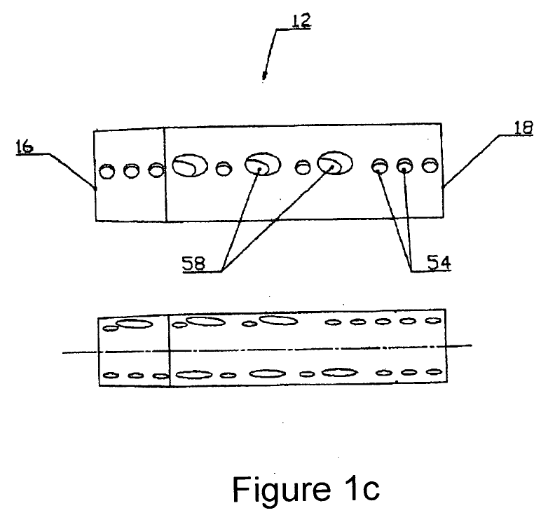

Reference is now made to FIGS. 1a-c illustrating views of a nail-cage implant in accordance with a preferred embodiment of the present invention. The nail cage implant is adapted to be incorporated in the hybrid interlocking fixation of the present invention. Nail-cage 12 is a hollow space tubular member having a triangular profile that is provided with sharp end 16 and blunt end 18 and a hollow space 55 that passes through the nail. Nail-cage 12 is adapted to be inserted to the femoral neck by forcing it into the bone through sharp end 18. Nail-cage 12 is preferably made of a biocompatible metal such as titanium. The nail-cage can be left in place in case of bone grafting or removed after the bone is healed; however, the nail-cage as well as the compression screws that pass through it may be made from a biodegradable material.

It is preferable to provide the wall of nail-cage 12 with a plurality of relatively small holes 54 so as to allow the bone to grow into the nail-cage 12 in order to allow contact and bone growing together with the grafting materials. It is optional to introduce autologous bone graft or osteoconductive-osteoinductive materials into nail-cage 12 (not shown in the Figures) so as to encourage bone grow.

Nail-cage 12 is provided with passages and preferably three passages 56 that pass through the wall of the tube and extend from sharp end 16 to blunt end 18. Passages 56 are provided with openings at both the sharp end and the blunt end through which compression lag screws as will be shown herein after may be screwed after the implantation of nail-cage 12. The nail cage of the present invention has the properties of compression and sliding when it is combined with the three lag screws.

The nail cage of the present invention is inserted to within the femoral neck through a drilling hole that is performed before the nail-cage insertion.

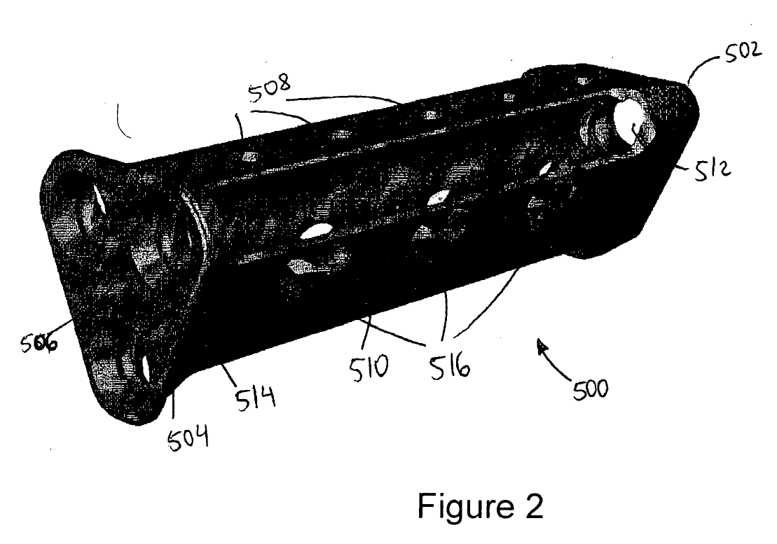

Reference is now made to FIG. 2 illustrating an isometric view of a nail-cage implant in accordance with a preferred embodiment of the present invention. The nail cage implant is adapted to be incorporated in the hybrid interlocking fixation of the present invention. Nail-cage 500 is a cage-like member having a triangular profile that is provided with sharp end 502 and blunt end 504 and a hollow space 506 that passes through the nail-cage. Nail-cage 500 is adapted to be inserted to the femoral neck by forcing it into the bone through sharp end 502. Nail-cage 500 is preferably made of a biocompatible metal such as titanium. The nail-cage can be left in place in case of bone grafting or removed after the bone is healed; however, the nail-cage as well as the compression screws that pass through it (the compression screws are not shown in FIG. 1) may be made from a biodegradable material.

Nail-cage 500 is preferably provided with a plurality of relatively small holes 508 so as to allow the bone to grow into nail-cage 500 in order to allow contact and bone growing together with the grafting materials that are preferably provided within hollow space 506. It is optional to introduce autologous bone graft or osteoconductive-osteoinductive materials into nail-cage 506 (not shown in the Figures) so as to encourage bone grow.

Nail-cage 500 is provided with passages and preferably three passages 510 that pass through nail-cage 500 and extend from sharp end 502 to blunt end 504. Passages 510 can be closed (as shown in nail-cage 10) or partially open passages that are provided with openings at both sharp end 502 and blunt end 504 through which compression lag screws as will be shown herein after may be screwed after the implantation of nail-cage 500. The nail cage of the present invention has the properties of compression and sliding when it is combined with the three lag screws. The passages are partially open so that when a screw is pined within the passage, it is partially exposed.

It should be noted that at least two passages for nails should be provided in order to prevent any rotational movement of the nail cage within the femoral head. As shown in FIGS. 1 and 2, three passages are the best mode of operation; however, any other number of passages can be applied.

Nail-cage 500 is further provided with bores 516 that are preferably provided in a row on one side of the nail-cage and extend to the opposite side of the cage wherein bores 516 are adapted to receive pin screws for external fixation as will be shown herein after. Bores 516 are provided with thread so as to allow compatible screws to be screwed through them.

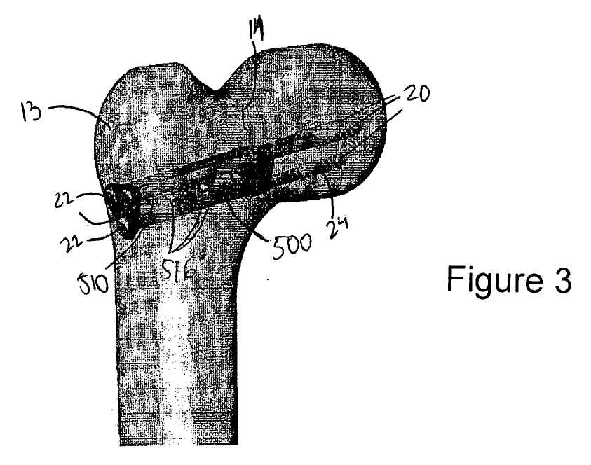

Reference is now made to FIG. 3 illustrating an isometric view of the nail-cage implant fixed in an internal fixation in accordance with a preferred embodiment of the present invention. Nail-cage 500 is implanted within a femoral bone 13 while preferably three lag screws 20 are interlocked within nail-cage 500 while passing through passages 510. Compression screws 20 (lag screws) are adapted to pass through nail-cage 500 from its blunt end 504 to its sharp end 502 so that the screws are extended proximally through the sharp end and screwed inside the bone. The extension of the screws beyond nail-cage 500 provides minimal metal volume penetration to the femoral head using only the three lag screws up to the subchondral bone. Minimal metal volume penetration prevents or avoids further damage to the vascularity in the femoral head. Each compression screw 20 is provided with a screw head 22 that prevents the screw from totally advancing into nail-cage 500. Compression screws 20 are used to compress the fracture so as to assure fast healing. The nail cage combined with the compression screws are adapted to perform sliding motion as well as compression. The compression is performed from blunt end 504 of the nail and the screw heads, anchoring the lateral trochanteric cortical bone around the blunt end of the nail-cage. The extended portion of compression screws 20 is provided with thread 24 so as to facilitate the compression process. Nail-cage 500 and compression screws 20 provide the possibility to introduce chip bone grafting up to the fracture line due to the nail's unique and special structure. In the case the nail-cage is hollow space, the interior of nail-cage 500, hollow space 506, can be filled with bone grafting materials so as to promote rapid bone healing.

It is important to notice that nail-cage 500 can be implanted in order to fixate a fracture 11 in the femoral bone 13 using compression lag screws 20 without employing an external fixation, however, an external fixation is also available.

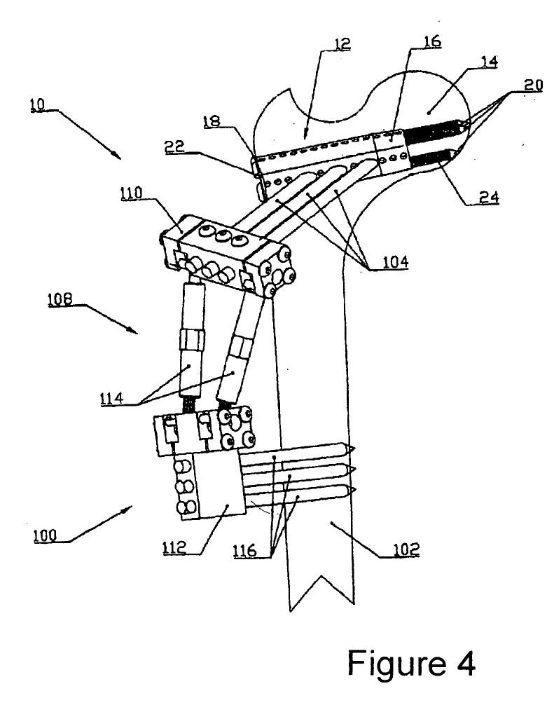

Reference is now made to FIG. 4 illustrating an isometric view of a hybrid interlocking proximal femoral fracture fixation in accordance with a preferred embodiment of the present invention, and its positioning in a femoral bone. The femoral bone and especially its neck are subjected to severe injuries that are difficult to fixate. Generally, the fracture fixation device of the present invention is a combination of internal and external fixation by interlocking system that connects the fractured upper fragment to the femoral shaft by a modular fixator. It is possible to correct the postoperative positioning in any case of varus or valgus deformity, rotation angulation shortening by distraction, compression, rotation, angulation, or translation. Causes for malpositioning can be related to unskilled operative technique as well as to low bone quality.

Hybrid device 10 comprises a nail-cage 12 that is adapted to be implanted inside the femoral neck 14. Nail-cage 12 is fixated to within femoral neck 14, however, nail-cage 500 can be employed in the same manner. The hollow space in nail-cage 12 allows drainage through the center hole of the nail so as to reduce the high pressure intracapsular hemarthrosis that develops from the fracture hemorrhage. The drainage reduces damage to the poor remaining vascularity and consequently reduces one of the common causes of avascular necrosis in the femoral head. Nail-cage 12 is inserted while crossing the fracture line by about 1-2 mm and allows bone grafting into the cage with autograft, allograft or other bone substitute.

Preferably three compression screws 20 are adapted to pass through nail-cage 12 from its blunt end 18 to its sharp end 16 so that the screws are extended proximally through the sharp end and screwed inside the bone. The extension of the screws beyond nail-cage 12 provides minimal metal volume penetration to the femoral head using only the three lag screws up to the subchondral bone. Minimal metal volume penetration prevents or avoids further damage to the vascularity in the femoral head. Each compression screw 20 is provided with a screw head 22 that prevents the screw from totally advancing into nail-cage 12. Compression screws 20 are used to compress the fracture so as to assure fast healing. The nail cage combined with the compression screws are adapted to perform sliding motion as well as compression. The compression is performed from blunt end 18 of the nail and the screw heads, anchoring the lateral trochanteric cortical bone around the blunt end of the nail-cage. The extended portion of compression screws 20 is provided with thread 24 so as to facilitate the compression process. Nail-cage 12 and compression screws 20 provide the possibility to introduce chip bone grafting up to the fracture line due to the nail's unique and special structure. In the case the nail-cage is hollow space, the interior of nail-cage 12 can be filled with bone grafting materials so as to promote rapid bone healing.

Nail-cage 12 is inserted into the femoral neck by minimal invasive procedure. The optimal angle of penetration of the nail-cage and the screws is between 95 to 115 degrees in respect of the femoral axis. In this way, the inferior screw of compression screws 20 is positioned so that it slightly touches the strong cortical bone of the calcar femori. All three compression screws 20 penetrate only the inferior half of the femoral head, preferable distally of the teres ligament vascularity, avoiding damage to the capilar spongeous intraosseous circulation and further reduce the possibility of avascular necrosis.

External fixator 100 connects nail-cage 12 to the distal bone fragment 102. External fixator 100 comprises preferably six pin-screws from which three pin-screws 104 are pined into nail-cage 12 and additional three pin screws 106 are pined in distal bone fragment 102. All six pin-screws, 104 and 106, are interconnected in a connecting member 108 comprising an upper clamp 110, which clamps pin-screws 104, and a lower clamp 112, which clamps pin-screws 106. Connecting member 108 further comprises preferably two rotating screwing rods 114 that connect upper clamp 110 and lower clamp 112. Rotating screwing rods 114 have changeable length and connect the clamps in a manner that allows a certain degree of freedom in the positioning of the clamps in respect with each other.

It should be noted that the external fixator is provided with poly-axial joints that allows 3-D correction ability of the fixator during and after the operation.

As mentioned herein before, bone grafting materials are inserted within the hollow space in the nail-cage or in any other space in the cage. It should be emphasized that insertion of the bone grafting materials can be performed before the insertion of the nail-cage into the femoral neck through a drilled hole.

The device of the present invention is intended to be used for intracapsular and extracapsular (including subtrochanteric) femoral fractures.

One of the important features of the hybrid interlocking fixation apparatus of the present invention is that the procedure of the present invention is of minimal invasive surgery instrumentations, mainly based on using multiple tissues penetrations by pin-screws. This fact facilitates the use of robotics for fast, exact, and controlled operative steps.

The external parts of the hybrid interlocking fixation apparatus are suitable for removal in the outpatient follow-up without anesthesia. The internal nail-cage and screws may be left in place. The nail-cage and screws can be made of bio-resorbable materials that are resorbed after some time as well as any other suitable material. The nail-cage and screws can be removed from the femoral neck after a certain time.

The parts of the fixator that are inserted into the body can be made with radiolucent materials so as to facilitate the x-ray imaging follow-up of the bone healing process.

It is optional to provide the device of the present invention with disposable parts so as to reduce the costs.

It should be clear that the description of the embodiments and attached Figures set forth in this specification serves only for a better understanding of the invention, without limiting its scope as covered by the following Claims.

It should also be clear that a person skilled in the art, after reading the present specification can make adjustments or amendments to the attached Figures and above described embodiments that would still be covered by the following Claims.

1. An internal fixation device for fixing an intracapsular fracture of a femoral neck, comprising:
a nail-cage member having a sharp end and a blunt end;
at least one hollow space extending from said sharp end to said blunt end;
at least two passage extending from said sharp end to said blunt end;
at least two screws adapted to pass through said at least two passages and extend beyond said sharp end;
thread provided in a portion of said at least two screws that is adapted to extend beyond said sharp end;
screw head provided in each of said at least two screws so as to prevent said at least two screws from being fully inserted into said at least two passages;
whereby said nail-cage member is implanted in the bone crossing the fracture line by at least 1 to 2 milimeters, and said at least two screws are inserted through said at least two passages so that compression of fragments of the bone is maintained in order to facilitate the healing process.

2. The internal fixation device as claimed in claim 1, wherein said nail-cage member is tube-like.

3. The internal fixation device as claimed in claim 1, wherein said nail-cage member has a profile such as a circular, oval, triangular, or rectangular or others.

4. The internal fixation device as claimed in claim 1, wherein nail-cage member is provided with holes.

5. The internal fixation device as claimed in claim 1, wherein said at least one hollow space is filled with bone grafting materials so as to promote bone healing.

6. The internal fixation device as claimed in claim 1, wherein said at least two passages are partially open.

7. The internal fixation device as claimed in claim 1, wherein three lag screws are provided to correspond three passages that are provided in said nail-cage member.

8. The internal fixation device as claimed in claim 7, wherein said nail-cage member is inserted to the femoral neck in an angle of preferably about 95-115 degrees in respect with an axial line of the femoral shaft so that an inferior screw of said three lag screws is positioned in a direction of an the inferior quadrant of the femoral head so as to slightly touch a the strong cortical bone of a calcar femori of the femoral neck.

9. The internal fixation device as claimed in claim 7, wherein said three screws are adapted to penetrate an inferior quadrant of the femoral head, preferable distally of the teres ligament vascularity.

10. The internal fixation device as claimed in claim 1, wherein said at least one hollow space is provided with bone or bone substitutes allowing bone grafting into said nail-cage member.

11. The internal fixation device as claimed in claim 1, wherein said fixation device is further interlocked with external fixator.

12. A method for fixing an intracapsular fracture of a femoral neck, the method comprising:
providing an internal fixator comprising
a nail-cage member having a sharp end and a blunt end;
at least one hollow space provided in said nail-cage member wherein said at least one hollow space extends from said sharp end to said blunt end;
and at least two passage extending from said sharp end to said blunt end;
performing 2-4 cm long skin incision at the trochanter region after fracture reduction;
inserting said internal fixator to the femoral neck preferably in an angle of about 95-115 degrees in respect with an axial line of the femoral shaft so that an inferior passage of said at least two passages is adapted to be positioned in a direction toward the inferior quadrant of the femoral head so as to slightly touch a strong cortical bone of the calcar femori, wherein said nail-cage member is crossing the fracture line by at least 1 to 2 milimeters;
providing at least two screws, each screw comprising:
thread provided in a portion that is adapted to extend beyond said sharp end;
screw head adapted to prevent the screw from being fully inserted into said nail-cage;
screwing said at least two screws so as to pass through said at least two passages and extend beyond said sharp end.

13. The method as claimed in claim 12, further comprising inserting bone grafting materials to within said at least one hollow space.

14. The method as claimed in claim 12, further comprising:
providing at least two bores in said nail-cage member and at least two opposite bores on opposite side of said nail-cage member;
interlocking at least two pin screws through the bores;
nailing at least two pin screws in a distal bone fragment;
providing a connecting member adapted to secure all pin screws together.

15. The method as claimed in claim 12, further comprising correcting, reducing and fixating in intra and post-operative period.
